(19) United States
(12) Patent Application Publication
(10) Pub. No.: US 2024/0116861 A1
(43) Pub. Date: Apr. 11, 2024

(54) DITHIAPOLYETHER DIOL, METHOD FOR PRODUCING SAME, SNAG PLATING SOLUTION CONTAINING DITHIAPOLYETHER DIOL, AND METHOD FOR FORMING PLATING FILM WITH USE OF SNAG PLATING SOLUTION

(71) Applicant: MITSUBISHI MATERIALS CORPORATION, Tokyo (JP)

(72) Inventor: Koji Tatsumi, Toyonaka-shi (JP)

(73) Assignee: MITSUBISHI MATERIALS CORPORATION, Tokyo (JP)

(21) Appl. No.: 17/768,312

(22) PCT Filed: Dec. 11, 2020
(86) PCT No.: PCT/JP2020/046221

§ 371 Date: Apr. 12, 2022

(30) Foreign Application Priority Data
Dec. 12, 2019 (JP) 2019-224523
Dec. 8, 2020 (JP) 2020-203251

(51) Int. Cl. C07C 323/12; C25D 3/56

(52) U.S. Cl. CPC C07C 323/12; C25D 3/56

(57) ABSTRACT

This dithiapolyether diol has a halogen content of less than 10 ppm and a purity of 80% or more and is represented by the following general formula (1) or (2). In the general formula (1) or (2), x and y are arbitrary natural numbers

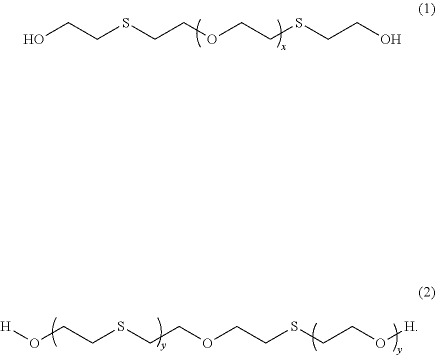

### TECHNICAL FIELD

The present invention relates to dithiapolyether diol, which is a kind of sulfide compound that is preferably used for SnAg plating solutions, and a method for producing the same. The present invention relates to a SnAg alloy plating solution for forming a SnAg plating film by an electroplating method. Furthermore, the present invention relates to a method for forming a plating film for semiconductor wafers or printed circuit boards using a SnAg plating solution.

The present application claims priority on Japanese Patent Application No. 2019-224523 filed on Dec. 12, 2019, and Japanese Patent Application No. 2020-203251 filed on Dec. 8, 2020, the contents of which are incorporated herein by reference.

### BACKGROUND ART

The present inventor has proposed a sulfide compound represented by the following general formula (21) as a sulfide compound that is used in tin alloy plating solutions (refer to Patent Document 1 (Claim 1, paragraph [0033])). In the formula (21), n is 1 to 3.

HO—CH₂CH₂—S—(CH₂CH₂—O—CH₂CH₂—S)ₙ—CH₂CH₂—OH (21)

This sulfide compound is obtained by dehydrating and condensing thiodiethanol (n=0) in a strong acid having a dehydrating action such as concentrated sulfuric acid or alkyl sulfonic acid. The value of n in the general formula (21) can be controlled by changing the reaction temperature, the reaction time and the purification conditions at this time.

Conventionally, 3,9-dithia-6-oxa-1,11-undecanediol (HOCH₂CH₂SCH₂CH₂OCH₂CH₂SCH₂CH₂OH) has been disclosed as a sulfide compound that is used for the development treatments of a silver halide photographic photosensitive material (for example, refer to Patent Document 2 (column 14, lines 18 to 28)). This compound is synthesized by the following method. 15.6 g of 2-mercaptoethanol, 14.3 g of bis-(2-chloroethyl) ether, and 10.6 g of sodium carbonate are dissolved in 50% ethanol. Then, the solution is refluxed for 20 hours, and the solvent is distilled away under reduced pressure. Next, the above-described compound is extracted with hot absolute ethanol and ethyl acetate. As a result, the above-described compound is synthesized. The targeted product (the above-described compound) is obtained by distillation.

However, since the sulfide compound described in Patent Document 1 is synthesized by dehydrating and condensing thiodiethanol (n=0) in a strong acid, not only a dimer formed by the dehydration and condensation of two molecules of “HOCH₂CH₂SCH₂CH₂OH” and “HOCH₂CH₂SCH₂CH₂OH” but also a multimer such as a trimer formed by additional condensation of one of the above-described molecules with this dimer or a tetramer are generated. Therefore, it is difficult to generate a highly pure sulfide compound suitable for tin alloy plating solutions. Due to this fact, in a case where this sulfide compound was used in a SnAg plating solution, there were cases where a Ag composition that was precipitated at the time of plating was not stable and the appearance and film thickness uniformity of plating films were impaired.

Regarding this respect, a multimer as described above is not generated in the reaction between 2-mercaptoethanol and bis-(2-chloroethyl) ether as described in Patent Document 2. However, in the reaction between 2-mercaptoethanol and bis-(2-chloroethyl) ether as described in Patent Document 2, in addition to the sulfide compound as a main reaction product, sodium chloride (NaCl) is generated as a by-product. Furthermore, a unreacted raw material or an organic impurity due to a side reaction also remains.

Due to these residues, the sulfide compound described in Patent Document 2 has a high chlorine content and a low purity. As described above, in a case where a sulfide compound having a high halogen content such as chlorine is used in a SnAg plating solution, there has been a problem in that a silver halide such as silver chloride is generated and the stability of the SnAg plating solution is likely to deteriorate due to the silver halide. In addition, in a case where a sulfide compound having a low purity is used in a SnAg plating solution, there has been a problem that platability, particularly, the appearance of plating films, is likely to deteriorate and the uniformity of the film thickness of the plating films is likely to deteriorate.

### PRIOR ART DOCUMENTS

### Patent Document

Patent Document 1: Japanese Patent No. 6432667
Patent Document 2: Japanese Examined Patent Application, Second Publication No. H4-28096

### DISCLOSURE OF INVENTION

### Problems to be Solved by the Invention

An object of the present invention is to provide dithiapolyether diol having a small halogen content and a high purity and a method for producing the same. Another object of the present invention is to provide dithiapolyether diol that, in the case of being used in a SnAg plating solution, improves the stability of the plating solution and the appearance and film thickness uniformity of plating films and a method for producing the same. That is, another object of the present invention is to provide a SnAg plating solution containing dithiapolyether diol and having favorable stability of the plating solution and a method for forming a plating film having a favorable appearance and favorable film thickness uniformity.

### Solutions for Solving the Problems

A first aspect of the present invention is dithiapolyether diol in which a halogen content is 10 ppm or less, a purity is 80% or more, and the dithiapolyether diol is represented by the following general formula (1) or (2). In the general formula (1) or (2), x and y are arbitrary natural numbers.

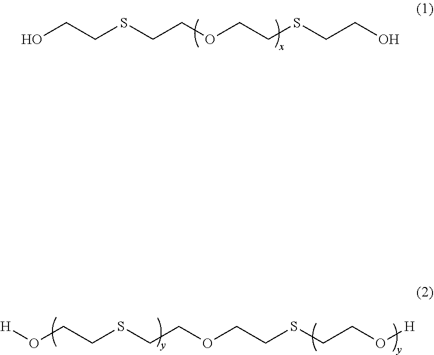

The purity is measured by the following method. In a high-performance liquid chromatography column (Prominence UFLC, manufactured by Shimadzu Corporation) filled with octadecyl silica (ODS) as a stationary phase, a gradient analysis is performed by changing the methanol concentration in stages from 10% to 100% using pure water as a mobile phase. The peak area of all detected components is regarded as 100%, and the area ratio of the peak area of the dithiapolyether diol is calculated. The calculated area ratio is regarded as the purity of the dithiapolyether diol. In a case where two or more kinds of dithiapolyether diol are contained, the area ratio of the peak area of the component having the largest peak area is regarded as the purity.

In the present specification, general formulae or formulae are structural formulae.

A second aspect of the present invention is the dithiapolyether diol according to the first aspect, in which a color number in Hazen units (APHA) as measured according to JIS K 0071-1 (1998) is 100 or less.

A third aspect of the present invention is a SnAg plating solution containing the dithiapolyether diol of the first or second aspect.

A fourth aspect of the present invention is a method for forming a plating film including a step of forming a plating film by using the SnAg plating solution of the third aspect.

A fifth aspect of the present invention is a method for producing dithiapolyether diol including: either one of a step (a-1) of obtaining a reaction product liquid containing an alkali metal salt by mixing and heating an alcohol compound having a mercapto group at one end (raw material A), an ether compound having halogen groups at both ends (raw material B), and an alkaline aqueous solution, or a step (a-2) of obtaining a reaction product liquid containing an alkali metal salt by mixing and heating an ether compound having mercapto groups at both ends (raw material A), an alcohol compound having a halogen group at one end (raw material B), and an alkaline aqueous solution; a step (b) of separating an organic phase and a water phase that are contained in the reaction product liquid of the step (a-1) or the step (a-2); a step (c) of removing a halide ion and a metal ion by bringing the organic phase into contact with an ion exchange resin; and a step (d) of evaporating and removing an impurity in the organic phase by heating the organic phase from which the halide ion and the metal ion have been removed.

A sixth aspect of the present invention is the method for producing dithiapolyether diol according to the fifth aspect, further including, between the step (b) and the step (c), a step (b-1) of diluting the organic phase with an organic solvent in which an alkali metal salt is not soluble to separate the alkali metal salt from the organic phase.

A seventh aspect of the present invention is the method for producing dithiapolyether diol according to the fifth or sixth aspect, further including, between the step (b) and the step (c) or between the step (b-1) and the step (c), a step (b-2) of bringing the organic phase into contact with activated carbon.

An eighth aspect of the present invention is the method for producing dithiapolyether diol according to any one of the fifth to seventh aspects, in which the evaporation of the impurity in the organic phase in the step (d) is performed under reduced pressure.

### Effects of Invention

The dithiapolyether diol according to the first aspect of the present invention contains an oxygen atom “—O—” in the molecule in the above-described general formula (1) or (2) and thus has an effect on improvement in water solubility due to a hydrogen bond with water in the case of being used in a SnAg plating solution. In addition, the dithiapolyether diol contains a hydroxyl group “—OH” and thus acts as a hydrophilic group and has an effect on additional improvement in water solubility due to a hydrogen bond with water. In addition, since an ether bond “C—O—C” is present between S atoms, the stability of the dithiapolyether diol itself is excellent. Since at least two S atoms are contained, these S atoms cause a silver ion nobler than tin in plating baths to sufficiently form a complex. In addition, since the halogen content is 10 ppm or less which is small, a silver halide is rarely generated in the plating solution. Therefore, this SnAg plating solution is excellent in terms of electrolytic stability and temporal stability for a long period of time while both in use and in storage. In addition, since the purity is 80% or more, the amount of a generated by-product is small, and the dithiapolyether diol is appropriately adsorbed to the surfaces of plating electrodes. Therefore, the appearance and film thickness uniformity of plating films become favorable.

Since the dithiapolyether diol according to the second aspect of the present invention has a color number in Hazen units (APHA) of 100 or less, in a case where the dithiapolyetherdiol diol is used in a SnAg plating solution, the plating solution becomes transparent.

The SnAg plating solution of the third aspect of the present invention contains dithiapolyether diol and is thus excellent in terms of electrolytic stability and temporal stability as described above. In addition, the SnAg plating solution makes the appearance and film thickness uniformity of plating films favorable.

According to the forming method of the fourth aspect of the present invention, since the SnAg plating solution of the third aspect is used, it is possible to produce plating films having a favorable appearance and a uniform film thickness.

According to the production method of the fifth aspect of the present invention, the raw material (A), the raw material (B), and the alkaline aqueous solution are mixed to obtain a reaction aqueous solution containing an alkali metal salt. This reaction aqueous solution is separated into an organic phase and a water phase, and halogen and metal ions are removed from the separated organic phase. Next, the organic phase is heated to remove an impurity; and thereby, a targeted product is obtained. Therefore, dithiapolyether diol having a low halogen content and a high purity can be produced.

According to the production method of the sixth aspect of the present invention, before the separated organic phase is treated with an ion exchange resin, the organic phase is diluted with an organic solvent in which alkali metal salts are not soluble. Therefore, it is possible to greatly reduce the contents of the halide ion and the metal ion and to increase the purification efficiency of the ion exchange resin.

According to the production method of the seventh aspect of the present invention, the organic phase is brought into contact with activated carbon after the reaction product containing the alkali metal salt is separated into an organic phase and a water phase and before the halide ion is removed from the separated organic phase. Therefore, a coloring impurity component of the organic phase is adsorbed to and removed by the activated carbon, and it is possible to make the dithiapolyether diol transparent.

According to the production method of the eighth aspect of the present invention, the impurity in the organic phase is removed under reduced pressure; and thereby, the impurity can be easily evaporated from the organic phase and it is possible to obtain dithiapolyether diol having a higher purity.

### EMBODIMENTS FOR CARRYING OUT THE INVENTION

Hereinafter, methods for producing dithiapolyether diol according to first and second embodiments of the present invention will be described.

### First Embodiment

### Method for Producing Dithiapolyether Diol

(a-1) Step of Mixing and Heating Raw Material (A), Raw Material (B), and Alkaline Aqueous Solution

The production method of the first embodiment includes a step (a-1). In the step (a-1), an alcohol compound having a mercapto group at one end (raw material A), an ether compound having halogen groups at both ends (raw material B), and an alkaline aqueous solution are mixed and heated to obtain a reaction product liquid containing an alkali metal salt. That is, the raw material (A) is an alcohol compound having a mercapto group at one end (hereinafter, also referred to as the raw material (A1)). Examples thereof include 2-mercaptoethanol represented by the following formula (3).

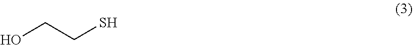

In addition, the raw material (B) is an ether compound having halogen groups at both ends (hereinafter, also referred to as the raw material (B1)). Examples thereof include compounds represented by the following formula (4) to formula (8). Table 1 below shows the ether compounds represented by the formula (4) to the formula (8) as the raw material (B) together with the raw material (A) of the formula (3).

TABLE 1

| Formula | |
|---|---|
| | Type of raw material (A) of first embodiment |
| (3) | 2-Mercaptoethanol |
| | Type of raw material (B) of first embodiment |
| (4) | Bis(2-chloroethyl) ether |
| (5) | 1,2-Bis(2-chloroethoxy)ethane |
| (6) | Diethylene glycol bis(2-chloroethyl) ether |
| (7) | Triethylene glycol bis(2-chloroethyl) ether |
| (8) | Bis-[2-[2-(2-chloroethoxy)ethoxy]ethyl] ether |

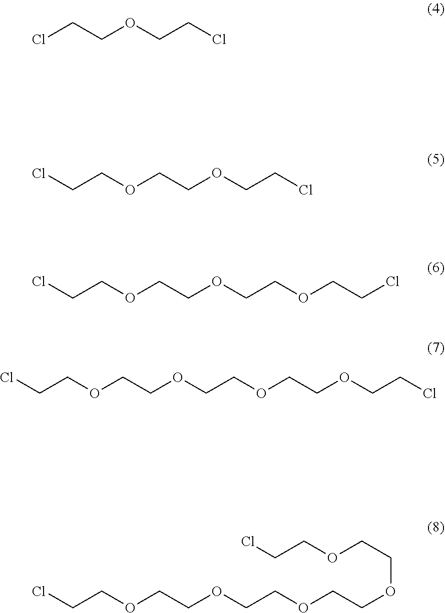 [SMILES: ClCCOCCCl ; ClCCOCCOCCCl ; ClCCOCCOCCOCCCl ; ClCCOCCOCCOCCOCCCl ; ClCCOCCOCCOCCOCCOCCCl]

Examples of the alkaline aqueous solution in the production method of the first embodiment include aqueous solutions of sodium hydroxide, potassium hydroxide, sodium carbonate, potassium hydrogen carbonate, and the like. Regarding the mixing of the raw material (A), the raw material (B), and the alkaline aqueous solution, for example, first, the raw material (A) is preferably uniformly mixed with the alkaline aqueous solution. This makes it possible to reliably perform the deprotonation of the raw material (A). In order to suppress the generation of heat during mixing, the alkaline aqueous solution is preferably cooled to a temperature of 0° C. to 10° C. before mixing. Next, the raw material (B) is preferably mixed with this mixture uniformly and heated. This heating is preferably performed by heating the mixture to a temperature of 40° C. to 100° C. in the atmosphere and holding the temperature for 3 hours to 24 hours. As a result, a dehalogenation reaction occurs, and a reaction product liquid containing dithiapolyether diol, which is the main reaction product, a halide alkali metal salt, which is a by-product, the unreacted raw material, and an organic impurity generated by a side reaction is obtained.

(b) Step of Separating Reaction Product Liquid into Organic Phase and Water Phase

The obtained reaction product liquid contains an organic phase and a water phase in a mixed state and is thus separated into the organic phase and the water phase. In a case where the amount of the reaction product liquid is small, the reaction product liquid is separated into the organic phase and the water phase with a separating funnel. In a case where the amount of the reaction product liquid is large, the reaction product liquid is separated into the organic phase and the water phase by centrifugation or the like. The reaction product liquid may be concentrated in order to facilitate separation into the organic phase and the water phase.

(b-1) Step of Diluting Organic Phase with Organic Solvent in which Alkali Metal Salts are not Soluble

It is preferable to collect the separated organic phase, add an organic solvent in which alkali metal salts are not soluble to the organic phase, and dilute the organic phase 2 to 10 times in terms of the volume ratio. That is, the organic solvent is preferably added such that the volume after dilution becomes 2 to 10 times the volume before dilution. As this organic solvent, isopropanol, toluene, and the like can be used. Since this makes it easy for the alkali metal salt to be precipitated or deposited, it is possible to obtain an organic phase having a small content of the alkali metal salt by the solid-liquid separation of the alkali metal salt.

(b-2) Step of Bringing Organic Phase into Contact with Activated Carbon

It is preferable to bring the organic phase of the step (b) or the organic phase that has undergone the step (b-1) into contact with activated carbon to make the colored organic phase with an unusual odor transparent and deodorize the organic phase. In a case where the amount of the organic phase is small, granular activated carbon is mixed with the organic phase to bring the organic phase into contact with the activated carbon. In a case where the amount of the organic phase is large, the organic phase is passed through a column filled with activated carbon to bring the organic phase into contact with the activated carbon. As the activated carbon, activated carbon, for example, “TAIKO K type” manufactured by Futamura Chemical Co., Ltd., “KURARAY COAL” manufactured by Kuraray Co., Ltd., and “purified SHIRASAGI” manufactured by Osaka Gas Chemicals Co., Ltd. can be used. The step (b-2) may be performed a plurality of times as necessary.

In a case where the activated carbon is mixed with the organic phase, the amount (g) of the activated carbon with respect to the amount (L) of the organic phase is preferably set to 1 g/L to 200 g/L. In a case where the organic phase is passed through the column filled with activated carbon, the organic phase is preferably passed through the column at a flow rate at which the linear velocity LV becomes 0.1 to 5.

(c) Step of Bringing Organic Phase into Contact with Ion Exchange Resin

The organic phase of the step (b), the organic phase that has undergone the step (b-1), or the organic phase that has undergone the step (b-2) is brought into contact with an ion exchange resin to remove the remaining halide ion and metal ion. In a case where the amount of the organic phase is small, a granular ion exchange resin is mixed with the organic phase to bring the organic phase into contact with the ion exchange resin. In a case where the amount of the organic phase is large, the organic phase is passed through a column filled with an ion exchange resin. Examples of the ion exchange resin include “A series” and “C series” manufactured by Purolite, “DUOLITE” manufactured by Sumika Chemtex Co., Ltd., “SK series”, “SA series”, “SM series”, and “SMUPB” manufactured by Mitsubishi Chemical Corporation can be used. The step (c) may be performed a plurality of times as necessary.

In a case where the ion exchange resin is mixed with the organic phase, the amount (g) of the ion exchange resin with respect to the amount (L) of the organic phase is preferably set to 1 g/L to 200 g/L. In a case where the organic phase is passed through the column filled with the ion exchange resin, the organic phase is preferably passed through the column at a flow rate at which the linear velocity LV becomes 0.1 to 5.

(d) Step of Removing Impurity in Organic Phase

The organic phase that has undergone the step (c) is heated; and thereby, the impurity in the organic phase is evaporated to remove the impurity. This deodorizes the organic phase having a residual unusual odor and increases the purity of dithiapolyether diol to be obtained. The evaporation of the impurity is preferably performed under conditions where a reduced pressure is 0.001 MPa to 0.01 MPa, a temperature is 40° C. to 100° C., and a time is 1 hour to 24 hours. This makes it easy for the impurity to be evaporated from the organic phase.

In the above-described embodiment, in the step (b-1), the organic phase is diluted with an organic solvent in which alkali metal salts are not soluble, and the alkali metal salt is precipitated and/or deposited to remove the alkali metal salt from the organic phase. However, it is also possible to remove the alkali metal salt from the organic phase by cleaning the organic phase with pure water.

[Dithiapolyether Diol]

Dithiapolyether diol produced by the above-described method of the first embodiment has a halogen content of 10 ppm or less and a purity of 80% or more and is represented by the above-described general formula (1). The halogen content is preferably 5 ppm or less, and the purity is preferably 90% or more.

Examples of the dithiapolyether diol of the first embodiment include the following formulae (9) to (13). The number of x in the general formula (1) is also shown. x is preferably within a range of 1 to 5 since it is easy to procure the raw materials.

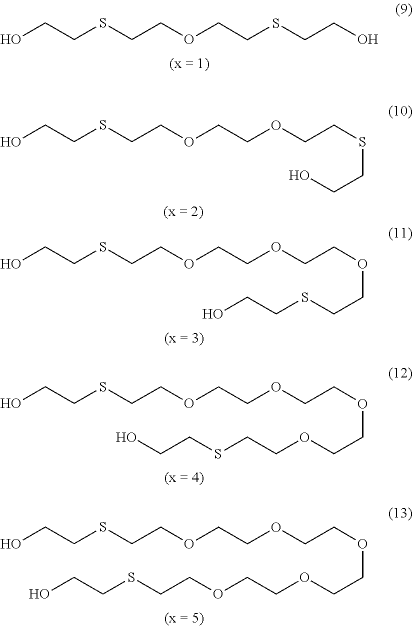

### Second Embodiment

### Method for Producing Dithiapolyether Diol

Next, a second embodiment of the present invention will be described. The production method of the second embodiment has a step (a-2). In the step (a-2), an ether compound having mercapto groups at both ends (raw material A), an alcohol compound having halogen groups at one end (raw material B), and an alkaline aqueous solution are mixed and heated to obtain a reaction product liquid containing an alkali metal salt. The production method of the second embodiment is the same as that of the first embodiment except that the raw material (A) and the raw material (B) in the second embodiment are different from those in the first embodiment.

In the production method of the second embodiment, the raw material (A) is an ether compound having mercapto groups at both ends (hereinafter, also referred to as the raw material (A2)). Examples thereof include bis(2-mercaptoethyl) ether represented by the following formula (14).

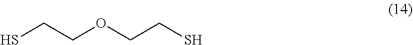

In addition, the raw material (B) is an alcohol compound having a halogen group at one end (hereinafter, also referred to as the raw material (B2)). Examples thereof include compounds represented by the following formula (15) to formula (17). Table 2 below shows the alcohol compounds represented by the formula (15) to the formula (17) as the raw material (B) together with the raw material (A) of the formula (14).

TABLE 2

| Formula | |
|---|---|
| | Substance name of raw material (A) of second embodiment |
| (14) | Bis(2-mercaptoethyl) ether |
| | Substance name of raw material (B) of second embodiment |
| (15) | 2-Bromoethanol |
| (16) | 2-(2-Chloroethoxy)ethane |
| (17) | 2-[2-(2-Chloroethoxy)ethoxy]ethanol |

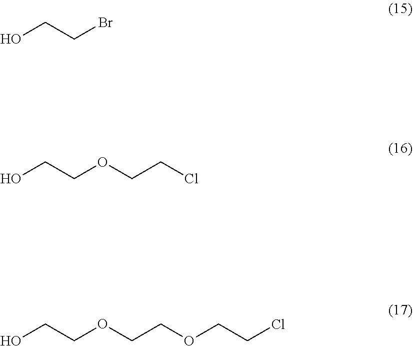

[Dithiapolyether Diol]

Dithiapolyether diol produced by the method of the second embodiment has the same characteristic values as the dithiapolyether diol produced by the method of the first embodiment. That is, this dithiapolyether diol has a halogen content of 10 ppm or less and a purity of 80% or more and is represented by the above-described general formula (2). The halogen content is preferably 5 ppm or less, and the purity is preferably 90% or more. Examples of the dithiapolyether diol of the second embodiment include the following formulae (18) to (20). The number of y in the general formula (2) is also shown. y is preferably within a range of 1 to 3 since it is easy to procure the raw materials.

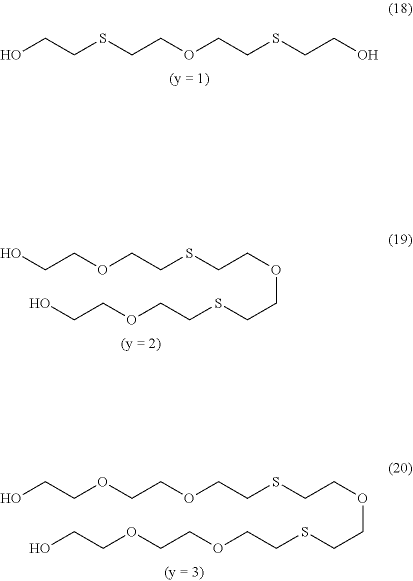

[SnAg Plating Solution and Method for Forming Plating Film Using this Plating Solution]

A SnAg plating solution of the present embodiment contains the dithiapolyether diol produced by the first and second embodiments and additionally contains a soluble Sn salt that dissolves in water to generate a divalent tin ion, a soluble Ag salt, and additives. The dithiapolyether diol acts as a complexing agent of Ag ions. Examples of the additives include an acid electrolyte (free acid), a surfactant, an antioxidant, a complexing agent for Sn, a pH adjuster, a brightening agent, and the like. This SnAg plating solution can be prepared by, for example, mixing a soluble tin salt, the above-described dithiapolyether diol and additives, and water.

As a method for forming a plating film using the SnAg plating solution of the present embodiment, an electroplating method is used. Examples of the plating film include plating films for semiconductor wafers and printed circuit boards. The liquid temperature of this SnAg plating solution at the time of plating is ordinarily 70° C. or lower and preferably 10° C. to 40° C. The current density at the time of forming a plating film by electroplating is within a range of 0.1 A/dm² or more and 100 A/dm² or less and preferably within a range of 0.5 A/dm² or more and 20 A/dm² or less.

The SnAg plating solution containing the dithiapolyether diol produced by the first and second embodiments is applied to an electronic component, which is an article to be plated; and thereby, a plating film can be formed on the electronic component. Examples of the electronic component include a printed circuit board, a flexible printed circuit board, a film carrier, a semiconductor integrated circuit, a resistor, a capacitor, a filter, an inductor, a thermistor, a crystal oscillator, a switch, a lead wire, and the like.

### EXAMPLES

Next, examples of the present embodiment will be specifically described together with comparative examples.

### Example 1

40 g (1 mol) of sodium hydroxide was dissolved in 200 mL of pure water, and the solution was cooled to 5° C. 78.1 g (1 mol) of 2-mercaptoethanol represented by the formula (3) as a raw material (A) was mixed with this sodium hydroxide aqueous solution and stirred with a stirrer to prepare a first mixture. Subsequently, 71.5 g (0.5 mol) of bis(2-chloroethyl) ether represented by the formula (4) as a raw material (B) was mixed with this first mixture and stirred with the stirrer to prepare a second mixture. This second mixture was heated up to 80° C. in the atmosphere and refluxed at 80° C. for 12 hours. As a result, the raw material (A) and the raw material (B) reacted with each other, and a reaction product liquid was obtained.

This reaction product liquid was transferred to a separating funnel, left to stand, and separated into two phases of an organic phase and a water phase. 2-isopropanol was mixed with the separated organic phase, and the organic phase was diluted 5 times in terms of the volume ratio. That is, 2-isopropanol was added such that the volume after dilution became 5 times the volume before dilution. A solid content precipitated or deposited by dilution was filtered; and thereby, the solid content was removed from the organic phase. Next, granular activated carbon (“purified SHIRASAGI” manufactured by Osaka Gas Chemicals Co., Ltd.) was mixed with the organic phase at a ratio of 10 g/L and stirred for 1 hour. After stirring, the mixed liquid was filtered to remove the activated carbon.

A granular ion exchange resin (“SMUPB” manufactured by Mitsubishi Chemical Corporation) was mixed with the organic phase from which the activated carbon had been removed at a ratio of 200 g/L and stirred for 1 hour. After stirring, the mixed liquid was filtered to remove the ion exchange resin. Subsequently, the organic phase from which the ion exchange resin had been removed was transferred to an eggplant flask, the pressure was reduced to 0.005 MPa, the organic phase was heated at 80° C. for 12 hours to evaporate an impurity in the organic phase; and thereby, a liquid final product was obtained.

Table 3 below shows production conditions (No. 1) of dithiapolyether diols in Example 1 and Examples 2 to 8 and Comparative Examples 1 and 2, which will be described below. That is, Table 3 shows each type of the raw material (A) and the raw material (B) used in Example 1 and Examples 2 to 8 and Comparative Examples 1 and 2, which will be described below. In Table 3, numbers for formulae correspond to the numbers of the formulae shown in the embodiments.

TABLE 3

| | Production conditions of dithiapolyether diol (No. 1) | | | |
|---|---|---|---|---|
| | Raw material (A) | | Raw material (B) | |
| | Formula | Type | Formula | Type |
| Example 1 | (3) | 2-Mercaptoethanol | (4) | Bis(2-chloroethyl) ether |
| Example 2 | (3) | 2-Mercaptoethanol | (5) | 1,2-Bis(2-chloroethoxy)ethane |
| Example 3 | (3) | 2-Mercaptoethanol | (6) | Diethylene glycol bis(2- |
| | | | | chloroethyl) ether |
| Example 4 | (3) | 2-Mercaptoethanol | (7) | Triethylene glycol bis(2- |
| | | | | chloroethyl) ether |
| Example 5 | (3) | 2-Mercaptoethanol | (8) | Bis-[2-[2-(2- |
| | | | | chloroethoxy)ethoxylethyl] ether |
| Example 6 | (3) | 2-Mercaptoethanol | (4) | Bis(2-chloroethyl) ether |
| Example 7 | (3) | 2-Mercaptoethanol | (4) | Bis(2-chloroethyl) ether |
| Example 8 | (3) | 2-Mercaptoethanol | (4) | Bis(2-chloroethyl) ether |
| Comparative | (3) | 2-Mercaptoethanol | (4) | Bis(2-chloroethyl) ether |
| Example 1 | | | | |
| Comparative | (3) | 2-Mercaptoethanol | (4) | Bis(2-chloroethyl) ether |
| Example 2 | | | | |

In addition, Table 4 below shows production conditions (No. 2) of the dithiapolyether diols in Example 1 and Examples 2 to 8 and Comparative Examples 1 and 2, which will be described below. That is, Table 4 shows the presence or absence of the production steps in Example 1 and the Examples 2 to 8 and Comparative Examples 1 and 2, which will be described. In Table 4, the signs of the steps correspond to the signs of the steps described in the embodiment.

TABLE 4

| | Production conditions of dithiapolyether diol (No. 2) | | | | | | | | |
|---|---|---|---|---|---|---|---|---|---|
| | Presence or absence of the production steps | | | | | | | | |
| | | (b) | (b-1) | (b-2) | (c) | | | | |
| | (a) | Separation of | Dilution with | Contact with | Contact with | (d) | | | |
| | Heating and | generated | organic | activated | ion exchange | Removal of | Final product | | |
| | mixing | liquid | solvent | carbon | resin | impurity | Formula | x | y |
| Example 1 | Present | Present | Present | Present | Present | Present (under | (9) | 1 | — |
| | | | | | | reduced | | | |
| | | | | | | pressure) | | | |
| Example 2 | Present | Present | Present | Present | Present | Present (under | (10) | 2 | — |
| | | | | | | reduced | | | |
| | | | | | | pressure) | | | |
| Example 3 | Present | Present | Present | Present | Present | Present (under | (11) | 3 | — |
| | | | | | | reduced | | | |
| | | | | | | pressure) | | | |
| Example 4 | Present | Present | Present | Present | Present | Present (under | (12) | 4 | — |
| | | | | | | reduced | | | |
| | | | | | | pressure) | | | |
| Example 5 | Present | Present | Present | Present | Present | Present (under | (13) | 5 | — |
| | | | | | | reduced | | | |
| | | | | | | pressure) | | | |
| Example 6 | Present | Present | Present | Present | Present | Present (under | (9) | 1 | — |
| | | | | | | normal | | | |
| | | | | | | pressure) | | | |
| Example 7 | Present | Present | Present | Absent | Present | Present (under | (9) | 1 | — |
| | | | | | | reduced | | | |
| | | | | | | pressure) | | | |
| Example 8 | Present | Present | Absent | Present | Present | Present (under | (9) | 1 | — |
| | | | | | | reduced | | | |
| | | | | | | pressure) | | | |
| Comparative | Present | Present | Present | Present | Absent | Absent | (9) | 1 | — |
| Example 1 | | | | | | | | | |
| Comparative | Present | Present | Present | Present | Present | Absent | (9) | 1 | — |
| Example 2 | | | | | | | | | |

### Examples 2 to 8 and Comparative Examples 1 and 2

In each of Examples 2 to 8 and Comparative Examples 1 and 2, as shown in Table 3, the type of the raw material (A) and the type of the raw material (B) were the same as or changed from those of Example 1. In addition, as shown in Table 4, a step (a-1), a step (b), a step (b-1), a step (b-2), a step (c), and a step (d) were performed or not performed.

In Example 6, the removal of the impurity in the step (d) was performed “under normal pressure”. In Example 7, the contact with the activated carbon in the step (b-2) was not performed. In Example 8, the dilution with the organic solvent in the step (b-1) was not performed. Instead, the organic phase was diluted 5 times in terms of the volume ratio using pure water, and the contact with the ion exchange resin in the step (c) was repeated 10 times. In Comparative Example 1, the contact with the ion exchange resin in the step (c) and the removal of the impurity in the step (d) were not performed. In Comparative Example 2, the removal of the impurity in the step (d) was not performed. Except what has been described above, in Examples 2 to 8 and Comparative Examples 1 and 2, final products were produced in the same manner as in Example 1.

### Example 9

40 g (1 mol) of sodium hydroxide was dissolved in 200 mL of pure water, and the solution was cooled to 5° C. 69.1 g (0.5 mol) of bis(2-mercaptoethyl) ether represented by the formula (14) as a raw material (A) was mixed with this sodium hydroxide aqueous solution and stirred with a stirrer to prepare a third mixture. Subsequently, 124.9 g (1 mol) of 2-bromoethanol represented by the formula (15) as a raw material (B) was mixed with this third mixture and stirred with the stirrer to prepare a fourth mixture. The subsequent steps were performed in the same manner as in Example 1; and thereby, a final product was obtained.

In addition, Table 5 below shows production conditions (No. 1) of the dithiapolyether diols in Example 9 and Examples 10 and 11, which will be described below. That is, Table 5 shows each type of the raw material (A) and the raw material (B) used in Example 9 and Examples 10 and 11, which will be described below. In Table 5, numbers for formulae correspond to the numbers of the formulae shown in the embodiments.

TABLE 5

| | Production conditions of dithiapolyether diol (No. 1) | | | |
|---|---|---|---|---|
| | Raw material (A) | | Raw material (B) | |
| | Formula | Type | Formula | Type |
| Example 9 | (14) | Bis(2-mercaptoethyl) ether | (15) | 2-Bromoethanol |
| Example 10 | (14) | Bis(2-mercaptoethyl) ether | (16) | 2-(2-Chloroethoxy)ethane |
| Example 11 | (14) | Bis(2-mercaptoethyl) ether | (17) | 2-[2-(2- |
| | | | | Chloroethoxy)ethoxy]ethanol |

In addition, Table 6 below shows production conditions (No. 2) of the dithiapolyether diols in Example 9 and Examples 10 and 11, which will be described below. That is, Table 6 shows the presence or absence of the production steps in Example 9 and Examples 10 and 11, which will be described below. In Table 6, the signs of the steps correspond to the signs of the steps described in the embodiment.

TABLE 6

| | Production conditions of dithiapolyether diol (No. 2) | | | | | | | | |
|---|---|---|---|---|---|---|---|---|---|
| | Presence or absence of the production steps | | | | | | | | |
| | | (b) | (b-1) | (b-2) | (c) | | | | |
| | (a) | Separation of | Dilution with | Contact with | Contact with | (d) | | | |
| | Heating and | generated | organic | activated | ion exchange | Removal of | Final product | | |
| | mixing | liquid | solvent | carbon | resin | impurity | Formula | x | y |
| Example 9 | Present | Present | Present | Present | Present | Present (under | (18) | — | 1 |
| | | | | | | reduced | | | |
| | | | | | | pressure) | | | |
| Example 10 | Present | Present | Present | Present | Present | Present (under | (19) | — | 2 |
| | | | | | | reduced | | | |
| | | | | | | pressure) | | | |
| Example 11 | Present | Present | Present | Present | Present | Present (under | (20) | — | 3 |
| | | | | | | reduced | | | |
| | | | | | | pressure) | | | |

### Examples 10 and 11

In each of Examples 10 and 11, as shown in Table 5, the type of the raw material (A) and the type of the raw material (B) were the same as or changed from those of Example 9. In addition, as shown in Table 6, a step (a-2), a step (b), a step (b-1), a step (b-2), a step (c), and a step (d) were performed. In Examples 10 and 11, final products were produced in the same manner as in Example 9.

<Comparison Test and Evaluation>

The dithiapolyether diols, which were 13 types of the final products obtained in Examples 1 to 11 and Comparative Examples 1 and 2, were used as samples, and, for these samples, the halogen content, the purity, and the tone (color number in Hazen units) were measured and evaluated by the following methods. In addition, SnAg plating solutions were prepared using these dithiapolyether diols, and a plating test was performed to evaluate the stability of the plating solutions and the appearance and film thickness uniformity of plating films. These results are shown in Table 7.

TABLE 7

| | Halogen | | | Stability of | Appearance | Film thickness |
|---|---|---|---|---|---|---|
| | content | Purity | | plating | of plating | uniformity of |
| | (ppm) | (%) | Tone | solution | film | plating film |
| Example 1 | 6 | 95 | 20 | Good | Good | Good |
| Example 2 | 7 | 93 | 40 | Good | Good | Good |
| Example 3 | 5 | 92 | 40 | Good | Good | Good |
| Example 4 | 1 | 96 | 50 | Good | Good | Good |
| Example 5 | 8 | 95 | 20 | Good | Good | Good |
| Example 6 | 8 | 80 | 80 | Good | Fair | Fair |
| Example 7 | 10 | 92 | 240 | Good | Good | Good |
| Example 8 | 7 | 89 | 60 | Good | Good | Good |
| Comparative | 700 | 46 | 120 | Poor | Poor | Poor |
| Example 1 | | | | | | |
| Comparative | 8 | 50 | 70 | Poor | Poor | Poor |
| Example 2 | | | | | | |
| Example 9 | 4 | 93 | 70 | Good | Good | Good |
| Example 10 | 5 | 94 | 100 | Good | Good | Good |
| Example 11 | 6 | 95 | 30 | Good | Good | Good |

### (a) Halogen Content

The halogen content was calculated by quantifying the ion concentration of a halogen (F, Cl, Br, or I) in the sample by ion chromatography (Prominence HIC-SP, manufactured by Shimadzu Corporation).

(b) Purity

The sample (dithiapolyether diol) was introduced into a high-performance liquid chromatography column (Prominence UFLC, manufactured by Shimadzu Corporation) filled with octadecyl silica (ODS) as a stationary phase. A gradient analysis was performed by changing the methanol concentration in stages from 10% to 100% using pure water as a mobile phase. The peak area of all detected components was regarded as 100%, and the peak area of the sample was calculated in terms of the area ratio ((peak area of sample/peak area of all components)×100(%)). The calculated area ratio was regarded as the purity of the sample.

(c) Tone (Color Number in Hazen Units)

The sample (dithiapolyether diol) was separated into a glass cell, the color was measured using a spectrophotometer for color and turbidity (model number: TZ6000) manufactured by Nippon Denshoku Industries Co., Ltd. in a state where the sample was held at 40° C., and the color number in Hazen units (APHA) as the tone was obtained from the value.

(d) Plating Test

Methanesulfonic acid as a free acid, the sample (dithiapolyether diol), a nonionic surfactant (containing polyoxyethylene and polyoxypropylene added to ethylenediamine at a ratio of 50:50), and benzylidene acetone as a brightener were mixed with and dissolved in a tin methanesulfonate aqueous solution. Next, a silver methanesulfonate aqueous solution was further added to and mixed with the mixture. In addition, finally, ion exchange water was added to prepare a SnAg plating solution having the following composition. The mole ratio of the dithiapolyether diol to the amount of Ag in the SnAg plating solution having the following composition was 1:1. The tin methanesulfonate aqueous solution was prepared by electrolyzing a metallic tin plate in the methanesulfonic acid aqueous solution. The silver methanesulfonate aqueous solution was prepared by electrolyzing a metallic silver plate in the methanesulfonic acid aqueous solution.

(Composition of SnAg Plating Solution)
Tin methanesulfonate (as Sn²*): 50 g/L
Silver methanesulfonate (as Ag*): 0.005 mol/L
Methanesulfonic acid (as free acid): 200 g/L
Dithiapolyether diol: 0.005 mol/L
Nonionic surfactant: 10 g/L
Brightener: 10 mg/L
Ion exchange water: Balance

(d-1) Stability of Plating Solution

13 types of prepared SnAg alloy plating solutions were separately put into sealed glass bottles and stored at 25° C. for 1 month. After 1 month elapsed, the appearances of the solutions were visually observed, solutions that maintained transparency were evaluated as “good”, and solutions from which turbidity or a deposit was observed were evaluated as “poor”.

(d-2) Appearance of Plating Film

13 types of prepared SnAg alloy plating solutions were each put into a plating tank, a wafer having a pattern formed as a cathode was disposed in the solution, a Pt/Ti mesh plate was disposed as an anode, and a plating test was performed. As plating conditions, the liquid temperature was set to 25° C., the energization current was set to 4 A/dm², and the plating treatment time was set to 25 minutes. During the plating treatment, the plating solution was stirred with a cathode rocker. The appearance of a plating film formed in the pattern was observed with a laser microscope. Films having a surface roughness Ra of less than 2 μm on the surface of the plating film were evaluated as “good”, films having a surface roughness Ra of 2 μm or more and less than 5 μm were evaluated as “fair”, and films having a surface roughness Ra of 5 μm or more were evaluated as “poor”. The appearances of the plating films were evaluated with these three determination criteria.

(d-3) Film Thickness Uniformity of Plating Film

The uniformity of the film thickness of the plating film formed in the pattern was investigated. The maximum value (Tmax), the minimum value (Tmin), and the average value (Tave) of the film thicknesses of the plating film at 10 sites in a die were obtained, the film thickness uniformity was calculated with the following formula, and whether or not plating was performed uniformly was evaluated.

Uniformity of film thickness of plating film={(Tmax−Tmin)/(2×Tave)}×100(%)

Plating films having uniformity of the film thickness of the plating film of less than 5% were evaluated as “good”, plating films having uniformity of 5% or more and less than 10% were evaluated as “fair”, and plating films having uniformity of 10% or more were evaluated as “poor”. The uniformity of the film thickness of the plating film was evaluated with these three determination criteria.

As is clear from Table 4, Table 6, and Table 7, in Comparative Example 1, the contact with the ion exchange resin in the step (c) and the removal of the impurity in the step (d) were not performed. In Comparative Example 1, the halogen content was “700 ppm” which was extremely high, the purity was “46%” which was low, and the color number in Hazen units was “120” which was high. In addition, the stability of the plating solution and the appearance and film thickness uniformity of the plating film were all “poor”.

In addition, in Comparative Example 2, the removal of the impurity in the step (d) was not performed. In Comparative Example 2, the purity was “50%” which was low. In addition, the stability of the plating solution and the appearance and film thickness uniformity of the plating film were all “poor”.
In contrast, in Examples 1 to 5 and Examples 9 to 11, the step (a-1) or the step (a-2), the step (b), the step (c) and the step (d) in the fifth aspect of the present invention were performed. In Examples 1 to 5 and Examples 9 to 11, the obtained final products had a halogen content and a purity within the ranges shown in the first aspect of the present invention, and the stability of the plating solutions and the appearance and film thickness uniformity of the plating films, which were the results of the plating tests, in which the SnAg plating solution containing the final product was used, were all “good”.
In Example 6, the removal of the impurity in the step (d) was performed under normal pressure. In Example 6, the purity was “80%” which was slightly low, the tone was “80” which was high, and the appearance and film thickness uniformity of the plating film were each “fair”.
In Example 7, the contact with the activated carbon in the step (b-2) was not performed. In Example 7, the halogen content was “10 ppm” which was slightly high, and the color number in Hazen units of the tone was “240” which was extremely high. However, the stability of the plating solution, the appearance and film thickness uniformity of the plating film were all “good”.
In Example 8, in the step (b-1), the organic phase was not diluted with an organic solvent, but diluted with pure water. In Example 8, the tone was “60” which was slightly high, but the stability of the plating solution and the appearance and film thickness uniformity of the plating film were all “good”.

### INDUSTRIAL APPLICABILITY

The dithiapolyether diol of the present embodiment can be used in SnAg plating solutions for forming a part of electronic components such as solder plating films for semiconductor wafers or printed circuit boards.

1. Dithiapolyether diol,
wherein a halogen content is 10 ppm or less, a purity is 80% or more, and the dithiapolyether diol is represented by the following general formula (1) or (2), and
in the general formula (1) or (2), x and y are arbitrary natural numbers,

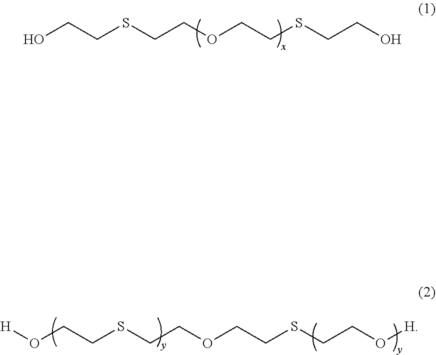

2. The dithiapolyether diol according to claim 1,
wherein a color number in Hazen units (APHA) as measured according to JIS K 0071-1 (1998) is 100 or less.

3. A SnAg plating solution comprising:
the dithiapolyether diol according to claim 1.

4-8. (canceled)
